(19) United States
(12) Patent Application Publication
(10) Pub. No.: US 2011/0160582 A1
(43) Pub. Date: Jun. 30, 2011

(54) WIRELESS ULTRASONIC SCANNING SYSTEM

(76) Inventors: Yongping Zheng, Hong Kong (CN); Xin Chen, Hong Kong (CN); James Chungwai Cheung, Hong Kong (CN); Junfeng He, Hong Kong (CN); Yanping Huang, Hong Kong (CN); Zhengming Huang, Hong Kong (CN)

(21) Appl. No.: 12/990,175

(22) PCT Filed: Apr. 28, 2009
(86) PCT No.: PCT/CN2009/000453

§ 371 Date: Jan. 24, 2011

(30) Foreign Application Priority Data
Apr. 29, 2008 (CN) 200810094380.4

(51) Int. Cl. A61B 8/00; G01S 15/00

(52) U.S. Cl. 600/437; 367/87

(57) ABSTRACT

A wireless ultrasonic scanning system comprises an ultrasonic sensor, a motor, an ultrasonic transceiver, a high-speed data sampling module, a motor controller and a master control module. The ultrasonic sensor, which moves in accordance with control of the motor controller, is mounted on the motor. The ultrasonic transceiver activates the ultrasonic sensor and amplifies a received ultrasonic signal. The high-speed data sampling module wirelessly transmits radio-frequency ultrasonic data to the master control module. The master control module sets a scanning mode, initiates a scanning process, and wirelessly transmits control signals and control parameters to the high-speed data sampling module.

### FIELD OF THE INVENTION

The present invention relates to an ultrasonic scanner, and more particularly, to a hand-held wireless ultrasonic scanning system.

### BACKGROUND OF THE INVENTION

A traditional ultrasonic scanner usually comprises a hand-held ultrasonic probe, a control box and a display unit. Generally, the control box is relatively larger and placed on a cart. The control box and the display unit are integrated for some systems. The probe and the control box are integrated for some systems. However, each module in those systems still transmits data information to each other via wires.

U.S. Pat. No. 6,780,154 “Segmented Handheld Medical Ultrasound System and Method” by Hunt et al. describes a segmented ultrasound system. In that system, a multi-purpose display unit communicates wirelessly with an integrated box including an ultrasonic probe and a control box. The data from an integrated ultrasonic sensor and the control box is in the format of video. The multi-purpose device is only used to display video images generated in the integrated box, but is irrelevant to process of signal and image.

In addition, the ultrasonic scanner will be used more and more by a non-professional operator without experience of being strictly trained as its size becomes smaller. Therefore, it is very important to standardize the operation. An important parameter in an ultrasonic scanning is the measured pressure applied to a tissue by the probe, and that pressure can be measured by attaching a force sensor to the ultrasonic probe. However, proper communication protocols are required to send those data, thereby those data can be analyzed together with ultrasonic images and other quantized data extracted from ultrasonic images.

### SUMMARY OF THE INVENTION

The object of the present invention is to provide a three-dimensional ultrasonic imaging system with accurate measurement, simple steps and low cost.

The present invention provides a wireless ultrasonic scanning system comprising an ultrasonic sensor, a motor, an ultrasonic transceiver, a high-speed data sampling module, a motor controller and a master control module, wherein the ultrasonic sensor mounted on the motor can move in accordance with control of the motor controller, the ultrasonic transceiver activates the sensor and amplifies received ultrasonic signals, the high-speed data sampling module wirelessly transmits radio-frequency ultrasonic data to the master control module, and the master control module sets a scanning mode, initiates a scanning process, and wirelessly transmits a control signal and a control parameter to the high-speed data sampling module.

According to the wireless ultrasonic scanning system as described in preferred embodiments of the present invention, the ultrasonic sensor comprises at least one ultrasonic sensing unit or array.

According to the wireless ultrasonic scanning system as described in preferred embodiments of the present invention, it further comprises a pressure sensor attached to the ultrasonic sensor for measuring pressure applied to a tissue.

According to the wireless ultrasonic scanning system as described in preferred embodiments of the present invention, the pressure sensor digitalizes the pressure signal and wirelessly transmits it to the master control module.

According to the wireless ultrasonic scanning system as described in preferred embodiments of the present invention, the ultrasonic transceiver comprises a T/R switch, a Low Noise Amplifier, a Time Gain Control, a high-voltage driver, a high-voltage control and a pulse generator.

According to the wireless ultrasonic scanning system as described in preferred embodiments of the present invention, the motor controller comprises a Micro Control Unit and a motor driver.

According to the wireless ultrasonic scanning system as described in preferred embodiments of the present invention, the high-speed data sampling module comprises an Analog to Digital Converter, a FIFO, a Digital Signal Processor, and a standard wireless communication interface.

According to the wireless ultrasonic scanning system as described in preferred embodiments of the present invention, the standard wireless communication interface is Bluetooth, wireless USB, WiFi or Zigbee.

According to the wireless ultrasonic scanning system as described in preferred embodiments of the present invention, radio-frequency ultrasonic data includes the ultrasonic signal, energy-saving control signal, ultrasonic transmission control signal and motor control signal.

The advantage of the present invention lies in that radio-frequency ultrasonic data are wirelessly transmitted to a multi-purpose device, and the multi-purpose device processes and displays ultrasonic signals and images. Therefore, the cost of an ultrasonic scanner is lower while the performance of the ultrasonic scanner is improved. Since these multi-purpose devices are widely used everywhere, a household ultrasonic scanner based on the present invention can finally be achieved. Furthermore, the present invention also discloses employing a pressure sensor to measure a pressure applied on tissue when scanning, thereby standardizing the operation.

### DESCRIPTION OF THE DRAWINGS

FIG. 1 is a schematic diagram showing modules of a wireless ultrasonic scanning system according to a first specific embodiment of the present invention; and

FIG. 2 is a schematic diagram showing modules of a wireless ultrasonic scanning system according to a second specific embodiment of the present invention.

### DETAILED EMBODIMENTS OF THE INVENTION

The present invention provides a wireless ultrasonic scanning system, the system utilizes an existing wireless communication channel of a multi-purpose device having a display and a microprocessor so as to wirelessly transmit a radio-frequency ultrasonic signal from an ultrasonic control box, and the multi-purpose device may be a desktop computer, a notebook computer, a PDA, a UMPC, a mobile phone and a game machine, etc. The ultrasonic control box connects with an ultrasonic sensor or an apparatus comprising an ultrasonic control circuit and the ultrasonic sensor.

The radio-frequency ultrasonic signal transmitted by wireless communication is processed by the multi-purpose device to form an image, or allow quantized parameters about ultrasonic images to be extracted from tissue. The wireless data communication also includes sending a parameter for controlling an ultrasonic control circuit from the multi-purpose device. The wireless data communication further includes sending parameters about the amount of an A-line scan and about whether it is the first line or last line of the A-line scan when a B-mode scan is performed. In addition, the wireless data communication also includes other parameters, for example, pressure applied on a probe corresponding to A-line signals and B-mode images.

### Embodiment 1

Hereinafter, the present invention, which may be applied to A-mode, i.e., collecting ultrasonic signals from signal points, or B-mode, i.e., generating images through mechanical scanning of a single ultrasonic sensing unit, will be introduced taking an ultrasonic scanner having a single sensing unit for a specific embodiment of the present invention. The present invention can also be applied to an ultrasonic sensor adopting multiple ultrasonic sensing units or arrays.

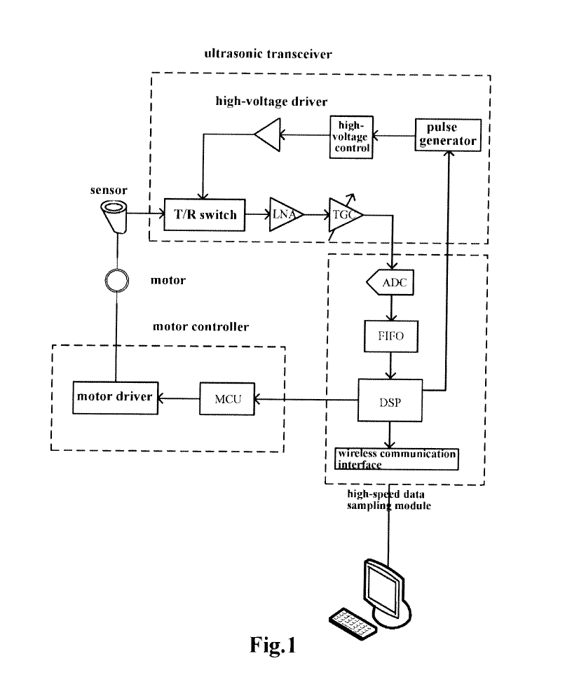

FIG. 1 is a modular schematic diagram of the wireless ultrasonic scanning system according to the first specific embodiment of the present invention, wherein the specific embodiment is a typical wireless ultrasonic scanning system adopting a single ultrasonic scanning unit and a motor for scanning.

As shown in FIG. 1, the wireless ultrasonic scanning system comprises an ultrasonic sensor, a motor, an ultrasonic transceiver, a high-speed data sampling module and a motor controller. Wherein, the ultrasonic transceiver includes a T/R switch, a LNA (Low Noise Amplifier), a TGC (Time Gain Control), a high-voltage driver, a high-voltage control and a pulse generator. The motor controller comprises a MCU (Micro Control Unit) and a motor driver. The high-speed data sampling module comprises an ADC (Analog to Digital Converter), a FIFO (First In First Out Memory), a DSP (Digital Signal Processor) and a standard wireless communication interface, such as Bluetooth, wireless USB, WiFi and Zigbee, etc, which is equipped in a multi-purpose device. The radio-frequency ultrasonic data may be wirelessly transmitted from the high-speed data sampling module to a computer. The computer can also wirelessly transmits a control signal and a control parameter to the high-speed data sampling module, such as the gain of the amplifier for ultrasonic signals and pressure signals, energy-saving control, ultrasonic transmission control and motor control, etc.

The ultrasonic sensor mounted on the motor can move in accordance with control of the motor controller. The ultrasonic transceiver activates the sensor and amplifies the received ultrasonic signal. The high-speed data sampling module acquires ultrasonic radio-frequency signals and communicates with the master control module via a USB or WiFi interface. The state of the motor controller may also be controlled by means of instructions from the master control module. The entire scanning process is controlled and synchronized by the master control module. The master control module sets a scanning mode (moving speed and moving track of the motor) and initiates the scanning process. In that process, the motor moves to different positions in accordance with a predetermined scanning mode. At each point, an A-line signal is captured and sent to the mater control module. After the scanning is completed, all the A-line data can be used for forming a B-mode image and stored in the master control module for further processing.

### Embodiment 2

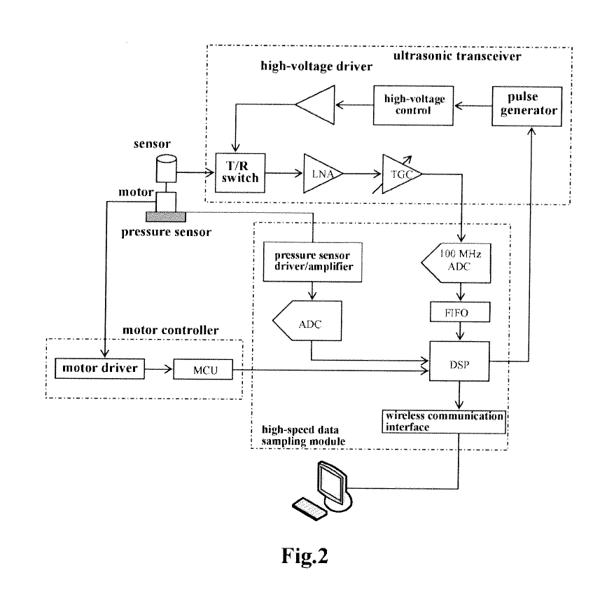

FIG. 2 is a modular schematic diagram of the wireless ultrasonic scanning system according to the second specific embodiment of the present invention. As shown in FIG. 2, the wireless ultrasonic scanning system comprises an ultrasonic sensor, a motor, a pressure sensor, an ultrasonic transceiver, a high-speed data sampling module, a motor controller and a master control module. The ultrasonic transceiver includes a T/R switch, a LNA (Low Noise Amplifier), a TGC (Time Gain Control), a high-voltage driver, a high-voltage control and a pulse generator. The motor controller comprises a MCU (Micro Control Unit) and a motor driver. The high-speed data sampling module comprises an ADC (Analog to Digital Converter), a FIFO (First In First Out Memory), a DSP (Digital Signal Processor) and a standard wireless communication interface, such as Bluetooth, wireless USB, WiFi and Zigbee. etc., which is equipped in a multi-purpose device. A radio-frequency ultrasonic data may be wirelessly transmitted from the high-speed data sampling module to a computer. Meanwhile, the pressure sensor is attached to the ultrasonic sensor or attached on a structure integrated by a sensor and a control box. The pressure sensor is used for measuring the pressure applied to the a tissue. When the ultrasonic scanner becomes smaller and smaller and is used more and more by an operator without experience of being strictly trained, the pressure information will be of vital importance since that pressure information can be used as an important reference for standard operation of the ultrasonic scanner. The pressure signal is digitalized by the pressure sensor and sent to the master control module together with the radio-frequency ultrasonic data.

The ultrasonic sensor mounted on the motor can move in accordance with control of the motor controller. The ultrasonic transceiver activates the sensor and amplifies the received ultrasonic signal. The high-speed data sampling module acquires ultrasonic radio-frequency signals and communicates with the master control module via a USB or a WiFi interface. The state of the motor controller may also be controlled by means of instructions from the master control module. The entire scanning process is controlled and synchronized by the master control module. The master control module sets a scanning mode (moving speed and moving track of the motor) and initiates the scanning process. In that process, the motor moves to different positions in accordance with a predetermined scanning mode. At each point, an A-line signal is captured and sent to the mater control module. After the scanning is completed, all the A-line data can be used for forming a B-mode image and stored in the master control module for further processing.

During the wireless communication, radio-frequency ultrasonic data and pressure data are arranged frame by frame. Each frame of data contains 1 byte or 2 bytes to indicate frame number, which can be used for representing the position of A-line in B-mode scan. In addition, that frame number information can also be used for checking whether there is frame loss. If the received frame data is discontinuous, that means one or more frames are not transmitted successfully. Following byte(s) indicating the frame number, there is 1 byte used for indicating the number of parameters related to ultrasonic signals, such as pressure. If there exist two parameters, the value of that byte is 2, and two parameters will be included in the data frame, each parameter may occupy 2 or 4 bytes. After that, A-line ultrasonic signals of 2 bytes may be set. Another 1 byte may be used for indicating the number of byte for each ultrasonic data point. Other information such as whether to use TGC may also be recorded in that byte. Finally, radio-frequency ultrasonic signals are set and each data point (or data dot) can occupy 1 byte or 2 bytes. The receiving end may receive different data bytes and read related data to perform subsequent process of signal and image.

Detail description to the present invention has been made above for those skilled in the art to understand the present invention. However, it can be conceived that other variations and modifications can be made without departing from the scope covered by the claims of the present invention, and those variations and modifications are all within the protection scope of the present invention.

### INDUSTRIAL APPLICABILITY

In the present invention, a radio-frequency ultrasonic data is wirelessly transmitted to a multi-purpose device, and the multi-purpose device processes and displays an ultrasonic signal and image. Therefore, the cost of an ultrasonic scanner is lower while the performance of the ultrasonic scanner is improved. Since these multi-purpose devices are widely used everywhere, a household ultrasonic scanner can finally be achieved based on the present invention. Furthermore, the present invention also discloses employing a pressure sensor to measure pressure applied to a tissue when scanning, thereby standardizing the operation.

What is claimed is:

1. A wireless ultrasonic scanning system, characterized by comprising an ultrasonic sensor, a motor, an ultrasonic transceiver, a high-speed data sampling module, a motor controller and a master control module, wherein the ultrasonic sensor mounted on the motor can move in accordance with control of the motor controller, the ultrasonic transceiver activates the sensor and amplifies received ultrasonic signals, the high-speed data sampling module wirelessly transmits radio-frequency ultrasonic data to the master control module, and the master control module sets a scanning mode, initiates a scanning process, and wirelessly transmits a control signal and a control parameter to the high-speed data sampling module.

2. The wireless ultrasonic scanning system of claim 1, characterized in that the ultrasonic sensor comprises at least one ultrasonic sensing unit or array.

3. The wireless ultrasonic scanning system of claim 1, characterized by further comprising a pressure sensor attached to the ultrasonic sensor for measuring a pressure applied to a tissue.

4. The wireless ultrasonic scanning system of claim 3, characterized in that the pressure sensor digitalizes the pressure signal and wirelessly transmits it to the master control module.

5. The wireless ultrasonic scanning system of claim 1, characterized in that the ultrasonic transceiver comprises a T/R switch, a Low Noise Amplifier, a Time Gain Control, a high-voltage driver, a high-voltage control and a pulse generator.

6. The wireless ultrasonic scanning system of claim 1, characterized in that the motor controller comprises a Micro Control Unit and a motor driver.

7. The wireless ultrasonic scanning system of claim 1, characterized in that the high-speed data sampling module comprises an Analog to Digital Converter, a FIFO, a Digital Signal Processor, and a standard wireless communication interface.

8. The wireless ultrasonic scanning system of claim 7, characterized in that the standard wireless communication interface is Bluetooth, wireless USB, WiFi or Zigbee.

9. The wireless ultrasonic scanning system of claim 1, characterized in that the radio-frequency ultrasonic data includes the ultrasonic signal, energy-saving control signal, ultrasonic transmission control signal and motor control signal.

10. The wireless ultrasonic scanning system of claim 1, characterized in that the master control module is a desktop computer, a notebook computer, a PDA, a UMPC, a mobile phone or a game machine.

11. The wireless ultrasonic scanning system of claim 1, characterized in that the radio-frequency ultrasonic data and pressure data are arranged frame by frame during the wireless transmission.

12. The wireless ultrasonic scanning system of claim 11, characterized in that each frame of data contains 1 byte or 2 bytes to indicate frame number during the wireless transmission.

13. The wireless ultrasonic scanning system of claim 11, characterized in that during the wireless transmission, each frame of data includes at least one byte for indicating the number of parameters related to ultrasonic signal, each parameter related to ultrasonic signals occupying 2 or 4 bytes.
